(19) United States
(12) Patent Application Publication
(10) Pub. No.: US 2005/0158848 A1
(43) Pub. Date: Jul. 21, 2005

(54) Method for immobolizing molecules on surfaces

(76) Inventors: Ulrich Sieben, Reute (DE); Ingo Freund, Vogtsburg-Oberrotweil (DE); Holger Klapproth, Freiburg (DE)

Correspondence Address: Patrick J O'Shea;O'Shea Getz & Kosakowski, Suite 912, 1500 Main Street, Springfield, MA, 01115, US

(21) Appl. No.: 10/511,192

(22) PCT Filed: Apr. 10, 2003
(86) PCT No.: PCT/EP03/03782

(30) Foreign Application Priority Data
Apr. 12, 2002 (DE) 102 16 446.0
Aug. 12, 2002 (DE) 102 36 925.9

(51) Int. Cl. C12M 1/34; B05D 3/00

(52) U.S. Cl. 435/287.2; 427/2.11

(57) ABSTRACT

In a method for immobilizing molecules on a surface, a layer of a hydrophobic, e.g., a non-swelling polymer, may be applied to the surface and molecules are immobilized on a surface of the polymer layer. The hydrophobic polymers may be polyimide or polystyrene, and the surface to which the polymer layer is applied may be made of an inorganic material, e.g., a semiconductor material (silicon), a semiconducting oxide (silicon dioxide), glass, nitride, or ceramic. Hydrophobic polymers such as polyimide or polystyrene can be applied to the surface of an inorganic support with conventional methods. Further, they electrically insulate the support with respect to the molecules applied to the surface of the polymer layer or substances associated with these molecules. Thus, electrical sensors and processor circuits can be integrated into the support without any detrimental effect on their function due to molecules and substances applied to the surface of the polymer layer.

### BACKGROUND OF THE INVENTION

The present invention relates in general to a method for immobilizing compounds, and in particular to the immobilization of molecules, such as for example biomolecules, on surfaces or solid supports.

Chip packaging techniques in which a plurality of ICs (integrated circuits) are housed in one package are known in the prior art. In the stacked-die structure, for example, interlayers made of polymers are frequently employed. The interlayers effect the connection of the ICs stacked one on another and at the same time help in mechanically protecting the top of the lower chip, which may be sensitive under some circumstances. In some factories the production of such layers is part of the mass production process and, in particular, the thicknesses of such layers can be accurately controlled within the range of a few microns and even less.

In many biochemical and biotechnological applications it is useful to immobilize biomolecules on solid supports. Crosslinkers that effect a connection between the inorganic layer and the biomolecules are commonly employed for bioconjugation on inorganic substrates. In European patent specification EP 1132739 B1, for example, a method is disclosed that serves, in bioconjugation, to bind molecules to inorganic substrates using crosslinkers, with silanes being among the candidates for such crosslinkers. Also proposed in EP 1132739 B1 is a linker system that can be used for the detection and isolation of biomolecules and as a component of a sensor chip or biochip or as a diagnostic instrument. Thus, in biochemical process sequences for example, an immobilized enzyme can be employed repeatedly. Furthermore, the immobilization of enzymes and other biomolecules is a key technology in the development of biocompatible implants.

A plurality of methods are known for binding molecules, for example biomolecules, to surfaces of supports. In the field of immunology, for example, polystyrene surfaces such as PolySorp and MaxiSorp are used to bind or conjugate biomolecules to surfaces.

From European patent specification EP 0646038 B1 it is known to produce passivated and stabilized porous supports and employ them for bioconjugation. These supports have a high reversible sorption capacity that essentially does not go along with nonspecific adsorption of molecules such as for example proteins, polysaccharides, or oligo- or polynucleotides.

German patent application DE 100 04 884 describes a method for emplacing biomolecules on surfaces using linker-like groups. The method includes bringing the polymer having linker-like groups into contact with a source of hydroxide ions, so that biomolecules such as for example heparin can be applied to substrate surfaces.

In relation to bioconjugation, two relevant issues are the number of molecules that bind to a well-defined surface, and the number of molecules that still display activity after the process of binding.

The known methods of bioconjugation have the disadvantage that linkers are used to bind the molecules to the support. The presence of the linkers disadvantageously reduces the activity of the bound molecules. Further, the known methods are time-consuming and expensive, because of the use of linkers or the corresponding analogs.

What is needed is a method that makes possible the conjugation or immobilization of biomolecules on a surface in an easy and economical manner, and wherein the activity of the bound molecules is preserved to the greatest extent.

### SUMMARY OF THE INVENTION

In a method for immobilizing molecules on a surface, a layer of a hydrophobic and, in a particular example, a non-swelling polymer may be applied to the surface and molecules are immobilized on a surface of the polymer layer.

Such hydrophobic polymers may, for example, be polyimide or polystyrene. The surface to which the polymer layer is applied may be made of an inorganic material such as for example a semiconductor material, in particular silicon, a semiconducting oxide, in particular silicon dioxide, glass, nitride, or ceramic.

Hydrophobic polymers such as polyimide or polystyrene have an advantage in that they can be applied to the surface of an inorganic support with conventional methods known in semiconductor technology. Further, they electrically insulate the support with respect to the molecules applied to the surface of the polymer layer or substances associated with these molecules. Thus, electrical sensors and processor circuits can be integrated into the support, which may be made for example of a semiconductor material, without any detrimental effect on their function due to molecules and substances applied to the surface of the polymer layer.

In the method, the surface of the support can be completely or only partly covered with the organic hydrophobic polymer. Typical masking processes found in the semiconductor industry may be used to hold back parts of the surface. In this way, electrical connection contacts (bonds) can later be emplaced on the support, for example on a chip. It is also possible to hold back parts of the surface for other reasons, thereby leaving regions of the surface, which may be inorganic if appropriate, uncovered or, conversely, to coat with the polymer only such definite locations of the surface on which molecules, for example biomolecules, can later adhere.

For immobilization, the polymer layer is, for example, brought into contact with organic molecules that can form a compound with the polymer layer. The act of bringing into contact is effected in such a manner that the molecules are bound in a positionally specific manner.

Sensor elements may preferably be integrated into the support under the surface to which the polymer layer is applied, so that measurements can be performed on the molecules immobilized on the surface of the polymer layer. These measurements can serve for example to characterize the properties of the biomolecules or chemical reactions that take place in their surroundings.

For example, antibodies can be well bound to surfaces of hydrophobic polymer layers made for example of a polyimide or a polystyrene, with which semiconductor articles or semiconductor layers serving as the support can easily be coated. This way, classical detection reactions such as for example ELISA reactions can be carried out subsequently.

Molecules are, in particular examples, peptides, proteins, genes and their fragments, nucleic acids, carbohydrate structures such as sugars, cells and their fragments, cell membrane constituents, and/or hormones. Microorganisms, cell extracts, ligands, antigens, antibodies, receptors, lectins, glycopeptides, and/or lipids can also be immobilized as the molecule on the surface of the polymer layer. The microorganisms can be live or dead microorganisms, live microorganisms including both growing and also resting cells. The microorganisms can be pre-immobilized by intracellular crosslinking of the cells on the support, the term “pre-immobilization” being understood to refer, for example, to all methods that can lead to fixation of the molecules or cells before immobilization by the method. Biopolymers such as for example polysaccharides or proteins or also synthetic polymers are, for example, expediently employed upon incorporation into a polymer matrix for pre-immobilization of the microorganisms.

Further, it is also possible, for example, to immobilize ligands as molecules on the surface of the polymer layer. Ligands are for example molecules, such as for example proteins or ions, that can be grouped about a central structure. Ligands can be monodentate and polydentate. The term “ligands” can, however, also be understood to refer to molecules that are bound at specific sites of macromolecules, for example substrates or coenzymes to a protein. The term “molecules” or “biomolecules” may also be understood to refer to antigens and/or antibodies. Antigens are, for example, all substances that can elicit an immune response. They can be natural or synthetic macromolecules foreign to the body, in particular proteins or polysaccharides, with a molecular weight of more than 2 kilodaltons, as well as surface structures of foreign particles. An antigen can have a high-molecular-weight part that serves as a substrate to usually a plurality of low-molecular-weight groups that govern the specificity of the immune response and the reaction of the antigens with the corresponding immunoglobulins. Antigens can be polyvalent and monovalent and thus can interact with one or with a plurality of antibody species.

It is also possible, for example, to immobilize antibodies on the support and not antigens. The term “antibodies” is understood, for example, to refer in particular to glycoproteins that interact specifically with one antigen. The interaction results in the formation of antigen-antibody complexes. Antibodies can be for example various groups of immunoglobulins. Antibodies can be immobilized as intact antibodies or as various fragments that can be generated for example through cleavage by various peptidases. The antibodies can be modified before, during or after immobilization on the support, for example by reduction, oxidation, or oligomerization. Further, it is also possible, for example, to use receptors as biomolecules. Receptors are for example proteins that interact with an extracellular signal molecule, for example a ligand, and activate or initiate certain functions through conformation changes, in particular via secondary messenger substances. Receptors can also, however, be special cells that receive stimuli and forward the corresponding items of information; examples of this are photoreceptors, chemoreceptors, thermoreceptors, and baroreceptors.

The surface to which the polymer layer is applied is preferably, for example, largely planar; that is, it is a surface with low roughness such as for example surfaces of semiconductor layers or semiconductor articles with integrated circuits (IC surfaces), which, however, can display local microscopic structures that may be suitable for example for the receiving of biomolecules.

The term “immobilization” or “pre-immobilization” may be understood to refer, for example, to all methods for restricting the mobility and solubility of molecules by chemical, biological, and/or physical operations, the term “pre-immobilization” pertaining, for example, to all methods for fixing molecules that are carried out before immobilization by the method. Immobilization and/or pre-immobilization can be effected by various methods, such as, for example, the binding of molecules to one another or to supports, entrapment in the network of a polymer matrix, or enclosure by membranes. As a result of immobilization, not only do the molecules become capable of repeated use, but after the process of interaction with the sample they can easily be separated again. They can be used in very much higher local concentrations and in continuous flow-through systems. The binding or immobilization of molecules on the support can be effected, for example, by direct attachment to the support and by crosslinking. Attachment to the support is effected in particular for example, by ionic, adsorptive, or covalent binding. Cross-linking is the linking of the molecules with one another or with other polymers. In immobilization by incorporation, the molecules are, for example, incorporated into gel structures or membranes before they are immobilized on the surface of the support.

There exist numerous possibilities for immobilizing molecules on the support. Immobilization may take place in such a manner that a well-defined position on the support can be assigned to every probe or molecule and every position on the support can be evaluated independently. It may, however, also be desirable for the places of deposition of various molecules or probes to overlap partially or fully or for biomolecule mixtures to be deposited. For example, immobilization can be effected by a method based on semiconductor technology. Essentially, molecules or biomolecules can be immobilized on the support in two fundamentally different ways:
(a) first, the molecules can be synthesized in situ at well-defined positions on the support by successive coupling of monomeric synthetic building blocks; and (b) second, previously synthesized biomolecules, biomolecules originating in libraries, or other molecules can be laid down and immobilized at well-defined positions of the support material, which is in particular functionalized. Both spotting and printing methods can be used for this purpose. The term “spotting” is understood, for example, to refer to methods in which liquid droplets, in which the molecules are situated, are laid down on the support, substantially round spots arising through surface interaction and drying. Other printing methods, however, also make it possible to lay down the molecules in well-defined areas on the surface of the support so that stable binding of the samples to the substrate surface of the molecules can take place with high coupling efficiency. Those known practices for immobilizing biomolecules on, for example, column materials can likewise be put to use to immobilize molecules on the support.

Selected methods for immobilization or pre-immobilization are for example contact tip printing, ring and pin printing, nanoelectric printing and nanopipetting, bubble jet printing, top-spot printing, micro contact printing, Micro Fluidic Networks methods, photolithographic activation methods, photoresist lithography, electrochemical focusing, and micro wet printing. All of these methods can be applied.

Inorganic surfaces including metal, polypropylene, Teflon, polyethylene, polyester, polystyrene, nitride, ceramic, and/or glass can be used as the support, as can IC (integrated circuit) surfaces, silicon, silicon dioxide, and other surfaces. Metals may all be compounds whose coherence arises from a crystal lattice. The boundary between metals and nonmetals is fluid, so that the elements Ce, Sn, As, and Sb are also metals as used herein. Metals may also include the metallic glasses, that is, materials that are in a metastable, largely amorphous state. Metallically conductive polymers are also metals as used herein. Metals advantageously display in particular, for example, good strength, good hardness and wear resistance, high toughness, and good electrical and thermal conductance. Polypropylenes are, for example, thermoplastic polymers of propylene. Polypropylenes are distinguished in particular by high hardness, resilience, rigidity, and thermal resistance. Polytetrafluoroethylenes, which advantageously display good thermoplastic qualities, are for example Teflon. Polyethylenes are made in particular, for example, by polymerizing ethylene by essentially two distinct methods, the high-pressure method and the low-pressure method. Polyethylenes produced by the high-pressure method advantageously display low density. The properties of supports that contain polypropylene are essentially determined by the nature of polyethylene as a partly crystalline hydrocarbon. Polyethylenes are advantageously practically insoluble in all usual solvents up to 60°. Advantageously, polar liquids such as alcohol, esters and ketones at room temperature cause little swelling of polyethylenes and thus of the support coating. Polyethylenes advantageously are completely indifferent to water, alkalies, and salt solutions as well as inorganic acids. Supports containing polyethylenes have, for example, very low permeability to water vapor. The support can, however, also expediently include polyesters. Polyesters are, for example, compounds produced by ring-opening polymerization of lactones or by polycondensation of hydroxycarboxylic acids or of diols and dicarboxylic acids or dicarboxylic acid derivatives. Polyesters also include, for example, polyester resins, polyesterimides, polyester rubbers, polyester polyols, and polyester polyurethanes. Polyesters are advantageously thermoplastics and have a marked material character. They are distinguished for example by high thermostability and can be processed into alloys with metals such as for example copper, aluminum, and magnesium.

It is also possible, however, for the support to contain ceramic. Ceramic as used herein is a collective term for compounds, in particular inorganic and predominantly nonmetallic compounds, containing more than 30 vol. % of crystalline materials. It is known that various ceramics or ceramic materials can be used as supports. Examples of these are stoneware, double-extruded tiles, laboratory porcelain, alumina ceramics, permanent magnet materials, silica brick, and magnesia brick. Among ceramic materials derived from clay, a distinction is made herein between coarse and fine materials, fine clay ceramic materials comprising earthenware, china, stoneware, and porcelain. Special ceramic materials such as vitrified ceramics, oxide ceramics, SiC brick and melt-cast brick can also advantageously be used as supports. The support can also preferably contain, for example, glass. The term “glass” as used herein refers to substances in the amorphous, noncrystalline solid state; that is, the glassy state can denote a frozen supercooled liquid or melt. Glasses are therefore inorganic or organic, chiefly oxidic melt products that have been converted to a solid state by an introduction process without the melt-phase components crystallizing out. Crystals, melts, and supercooled melts, for example, are also referred to as glasses as used herein. Glasses can be for example flat glass, laboratory glassware, lead crystal glass, fiberglass, optical glass fibers, and other glasses. It is also possible to use silicate-free glasses, for example phosphate glasses. The support can, however, also be such that optical glasses, that is, for example, glasses with special optical refractive indices, are used.

The surface of the support can be modified. Support modification can be effected in particular by biological, physical, and/or chemical operations. Examples of physical operations are polishing, etching, pickling, sandblasting, but also physical processes that lead to hardening, coating, heat treatment, production of protective skins, and the like. An example of surface treatment by biological action is overgrowth by microorganisms. A chemical modification of the surface of the supports includes for example treatment with acids, bases, metal oxides, and other agents. The support surface can be modified in such a manner that the molecules adhere especially well to the support or adhere in such a way that they are not modified disadvantageously in respect of their activity. Surface modification also includes, for example, coating with poly-L-lysines, aminosilanes, aldehyde silanes, epoxy groups, gold, streptavidin, reactive groups, polyacrylamide pads, immobilized nitrocellulose and/or activated aldehydes or agarose-aldehyde groups, by which in particular the following are bound: DNA, COO⁻ groups, NH₂ groups, biotin, thiol groups, and others. Support surface modification also includes, for example, treatment that leads to heightened stability and fracture strength. Classical surface modifications from histology, in particular for the immobilization of biomolecules, can also be performed.

In one exemplary embodiment, further semiconductor articles or semiconductor layers with integrated circuits or additional Microsystems are to be applied in certain sections of the surface of the polymer layer. Suitable as the polymer layer here is in a particular example a polyimide that is known for such applications in semiconductor technology. Polyimides are in particular polymers with high-temperature stability; they advantageously display excellent mechanical, thermal, and electrical properties. Previously known applications of polyimide in semiconductor technology include in particular buffer layers, passivation layers, bonding layers, and dielectric interlayers on the support. Polyimides are in particular applied in liquid form and then cured. In this curing step the polyimide advantageously acquires the desired qualities. The polyimide can be lithographically textured for the applications. Polyimide can also be used as an adhesion aid for potting material and as a buffer layer. The polyimide layer reduces, for example, the stress in silicon due to encapsulation and prevents cracks at the edges. The polyimide is cured under very uniform temperature conditions to prevent cracking in the polyimide and nonuniformities in color. Low oxygen values are advantageous, for example, to achieve good adhesion.

Polystyrene, which can also be employed as a polymer layer for immobilizing molecules in an embodiment, is a thermoplastic obtained chiefly by radical polymerization of styrene. The radical end of a growing polymer chain does not attack a double bond in the ring, because the benzene ring is an extraordinarily stable structure. A plurality of advantages for the employment of polystyrene stem from the fact that, for example, polystyrene is resistant to acids, alkalies and alcohol.

Further, the hydrophobic polymer is applied to the surface, for example, only in previously defined regions.

In a further exemplary embodiment, a positive and/or negative electric charge is imparted to the surface by plasma treatment; that is, the surfaces bear different charges at the most varied positions. Polymer materials in particular exist in various forms. The individual forms impose various requirements on the processing process. The accessibility of the surface to plasmas, for example, varies in dependence on the shaping of the surface. Plasma treatment of the polymer surface can advantageously increase the surface energy greatly and facilitate other processing methods. In plasma treatment, the ions and radicals of the plasma react with the polymer surface and there generate functional groups that advantageously determine the surface qualities of the polymer. In particular, positive or negative charging results in better wettability and/or better binding of the biomolecules.

In a further exemplary embodiment, UV-reactive molecules are covalently immobilized by irradiation with UV light. For example, photolabile protective groups on glass can be activated in positionally bound fashion for oligosynthesis through the use of light selectively passing through a photolithographic mask. The glass is then flooded with photolabile molecules, for example DNA bases, which bind to the defined, previously irradiated array positions. Different photolithographic masks are then correspondingly utilized for the next oligo bases in the sequences, and the procedure is repeated. Thus four masks are required for each base in the sample oligo (per position). Production can thus advantageously be effected directly from known sequence databases, a standard normalization being achieved in this way.

If hydrophobic molecules, in particular biomolecules, are applied by one of the above-cited methods, for example a printing method, to the surface of the hydrophobic polymer layer, these molecules adhere to this surface through a sufficiently well-known interaction.

In one exemplary embodiment of the method for immobilizing molecules on the surface of the polymer layer, the surface of the polymer layer is to be activated in an oxygen plasma at least in sectional fashion, for example with the use of a conventional masking technique. In this way aldehyde groups, carboxyl groups, or hydroxide groups are formed on the surface of the polymer layer. These groups are hydrophilic and make possible covalent bonds with biomolecules applied to these activated regions, for example by printing with a solution containing the molecules. These covalent bonds are so stable that the polymer layer with the molecules immobilized thereon can subsequently be boiled in soap without disrupting the bonds. The surface is preferably activated in an embodiment only in island manner by oxygen plasma treatment, runoff of the applied solution on the surface being limited by the polymer layer regions left hydrophobic and surrounding the “islands.”

These and other objects, features and advantages of the present invention will become more apparent in light of the following detailed description of preferred embodiments thereof.

### DETAILED DESCRIPTION OF THE INVENTION

### EXAMPLE 1

Silicon sensor chips with CMOS photodiodes are covered with a layer of approximately 100 to 200 nm of polystyrene in the spin coater. To this end, the chips are coated with 200 μL of a 0.1% (w/v) solution of polystyrene in toluene for one minute at 3000 rpm in the spin coater. Next, the sensor regions (photodiodes) are printed with a protein solution in a raster-like array. Antibodies in PBS buffer are employed. The antibodies are in each case used in a concentration of 5 μg/mL. A portion of the raster is printed with antibodies conjugated with fluorescent dyes. The antibodies are incubated overnight in a moist chamber at 4° C., and the unbound antibodies are then rinsed off with PBS buffer. After washing with distilled water, the success of the immobilization is verified with the aid of a fluorescence measuring instrument. The successful binding of the antibodies to the sensor regions is demonstrated by the fluorescence of the antibodies. Next the chip is sealed by application of a reaction chamber made of PMMA. The application of the reaction chamber is effected by binding of the PMMA to the polystyrene layer. The finished structure is further stabilized by the employment of a commercially available stabilizing reagent for proteins and is ready for use.

### EXAMPLE 2

Silicon sensor chips with CMOS photodiodes, already on the wafer, are coated with a 5 μm layer of polyimide. Next, the polyimide is coated with a copolymer of benzophenone methacrylate and acrylic acid. The supports can then be printed in simple fashion with biomolecules such as DNA (5 μM oligonucleotide in PBS buffer). Immobilization is effected by UV irradiation at 300 nm for approximately 10 minutes. The benzophenone of the copolymer forms radicals, which bond covalently to the polyimide coating as well as to the DNA. The same process can also be carried out with all other biomolecules such as proteins, in particular antibodies, peptides, sugars, lipids, and triglycerides as well as complex structures of the same.

Although the present invention has been shown and described with respect to several preferred embodiments thereof, various changes, omissions and additions to the form and detail thereof, may be made therein, without departing from the spirit and scope of the invention.

1. A method for immobilizing molecules on a surfaces of a support in which electrical sensors and processor circuits are integrated, the method comprising the steps of:
applying a layer of a hydrophobic polymer to the surface; and
immobilizing molecules on a surface of the layer.

2. The method of claim 1, wherein the polymer is from the group comprising a polyimide and a polystyrene.

3. The method of claim 1, wherein the polymer layer is applied to the surface in previously defined regions.

4. The method of claim 1, wherein an electric charge is imparted to the surface of the polymer layer, at least in sectional fashion, by plasma treatment.

5. The method of claim 1, wherein UV-reactive molecules are covalently immobilized by irradiation with UV light.

6. The method of claim 21, wherein the polymer layer is activated, at least in sectional fashion, in an oxygen plasma.

7. The method of claim 1, wherein a portion of the surface of the polymer layer is utilized for application with an integrated circuit.

8. A method for immobilizing molecules on a surface, comprising the steps of applying a layer of a hydrophobic polymer to the surface, and immobilizing molecules on a surface of the layer.

9. The method of claim 8, where the polymer is a polyimide.

10. The method of claim 8, where the polymer is a polystyrene.

11. The method of claim 8, further comprising the steps of forming at least one defined region on the surface, and applying the layer of a hydrophobic polymer to the at least one defined region on the surface.

12. The method of claim 8, where the polymer layer is activated in an oxygen plasma.

13. The method of claim 8, where UV-reactive molecules are covalently immobilized by irradiation with UV light.

14. The method of claim 8, where an electric charge is imparted to the surface of the polymer layer by plasma treatment.

15. The method of claim 8, where the molecules are biomolecules.

16. The method of claim 8, where the polymer comprises a non-swelling polymer.

17. The method of claim 8, where the surface to which the polymer layer is applied may comprise an inorganic material.

18. The method of claim 17, where the inorganic material is a semiconductor material.

19. The method of claim 18, where the semiconductor material comprises silicon.

20. The method of claim 17, where the inorganic material is a semiconducting oxide.
